(19) United States
(12) Patent Application Publication
(10) Pub. No.: US 2014/0017668 A1
(43) Pub. Date: Jan. 16, 2014

(54) COMPOSITIONS AND METHODS FOR DETECTING AND QUANTIFYING CIRCULATING TUMOR CELLS (CTCs)

(71) Applicants: Shawn Edward Lupold, Ellicott City, MD (US); Wasim Haider Chowdhury, Laurel, MD (US); Ping Wu, Baltimore, MD (US); Ronald Rodriguez, Glenwood, MD (US)

(72) Inventors: Shawn Edward Lupold, Ellicott City, MD (US); Wasim Haider Chowdhury, Laurel, MD (US); Ping Wu, Baltimore, MD (US); Ronald Rodriguez, Glenwood, MD (US)

(73) Assignee: THE JOHNS HOPKINS UNIVERSITY, Baltimore, MD (US)

(21) Appl. No.: 13/807,366

(22) PCT Filed: Jun. 30, 2011
(86) PCT No.: PCT/US11/42547

§ 371 Date: Oct. 1, 2013

Related U.S. Application Data
(60) Provisional application No. 61/359,862, filed on Jun. 30, 2010.

(51) Int. Cl. C12Q 1/68; C12N 15/86

(52) U.S. Cl. CPC C12Q 1/6897; C12N 15/86
USPC 435/5; 435/302.1

(57) ABSTRACT

The present invention relates to the field of virology. More specifically, the present invention relates to the use of viral constructs to detect and quantify circulating tumor cells. In one embodiment, the present invention provides an adenovirus construct comprising (a) a cell type specific promoter that drives adenoviral replication; and (b) at least one reporter gene incorporated into the viral Major Late Transcriptional Unit. In another embodiment, an adenovirus construct comprises (a) prostate selective pro-basin promoter operably linked to the El gene; and (b) prostate specific antigen enhancer operably linked to the probasin promoter.

### CROSS-REFERENCE TO RELATED APPLICATIONS

This application claims the benefit of U.S. Provisional Application No. 61/359,862, tiled Jun. 30, 2010; which is incorporated herein by reference in its entirety.

### STATEMENT OF GOVERNMENTAL INTEREST

This invention was made with U.S. government support under grant no. ROICA121153. The U.S. government has certain rights in the invention.

### FIELD OF THE INVENTION

The present invention relates to the field of virology. More specifically, the present invention relates to the use of viral constructs to detect and quantify target cells, namely, circulating tumor cells.

### BACKGROUND OF THE INVENTION

For over one hundred years it has been known that disseminated tumor cells exist in the blood of cancer patients. Ashworth, T. R., 14 MED. J. AUSTRALIA 146-69 (1869). Yet, there are no technologies capable of concurrently determining the level, viability, and origin of circulating tumor cells (CTCs). One can anticipate the impact such a technology would bring to cancer management. Early detection of viable disseminated tumor cells (at the time of diagnosis) would improve risk stratification and guide higher risk patients toward more aggressive therapies. Accurate quantification of CTCs could also indicate recurrence, stratify risk among metastatic patients, quickly monitor response to treatment, and potentially accelerate the clinical evaluation and approval of new cancer drugs. Considering that one quarter of all deaths in the United States can be attributed to cancer, primarily due to metastasis, there is a significant need for innovative strategies to detect, prevent, and treat metastatic cancer. See Jemal et al., 59(4) CA CANCER J. CLIN. 225-49 (2009).

### SUMMARY OF THE INVENTION

The present invention is based, in part, on the discovery that adenoviral reporter vectors can be used for the detection and quantification of viable disseminated tumor cells of specific tissue origin. This technology, called Circulating Tumor Cell Reporter Vectors (CTC-RVs), shifts from current approaches by not requiring special sorting equipment. manual microscopic examination, or analysis of the overwhelming blood cell population. Instead, in certain embodiments, a quantifiable reporter signal is secreted into the growth media, separate from the background of blood cells and debris. The present invention applies tissue-selective promoters and viral replication as distinct mechanisms for specificity and signal amplification.

Accordingly, in one embodiment, the present invention provides a virus construct comprising (a) a cell type specific promoter; and (b) at least one reporter gene incorporated into the viral Major Late Transcriptional Unit. In particular embodiments, a pharmaceutical composition comprises a virus construct. In another embodiment, the present invention provides an adenovirus construct comprising (a) a cell type specific promoter that drives adenoviral replication; and (b) at least one reporter gene incorporated into the viral Major Late Transcriptional Unit. In yet another embodiment, an adenovirus construct comprises a cell type specific promoter that drives adenoviral replication.

In a specific embodiment, an adenovirus construct comprises a prostate cancer cell specific promoter that drives adenoviral replication. An adenovirus construct may also comprise (a) a prostate cancer cell specific promoter that drives adenoviral replication; and (b) at least one reporter gene incorporated into the viral Major Late Transcriptional Unit.

In a more specific embodiment, the present invention provides an adenovirus construct comprising (a) the prostate selective probasin promoter operably linked to the E1 gene; and (b) the prostate specific antigen enhancer operably linked to the probasin promoter. An adenovirus construct can simply comprise a cell-type specific promoter operably linked to a reporter gene. In particular embodiments, a pharmaceutical composition comprises an adenovirus construct.

In another aspect, the present invention provides methods for detecting circulating tumor cells in a biological sample using the adenoviruses described herein. In one embodiment, the methods comprise (a) contacting an adenovirus construct of the present invention with the biological sample obtained from a patient; and (b) analyzing reporter gene activity to detect circulating tumor cells in the biological sample. In another embodiment, a method for detecting circulating tumor cells in a biological sample from a patient comprises the steps of (a) obtaining a biological sample from a patient, wherein the biological sample comprises a mixed cell population suspected of containing circulating tumor cells; (b) contacting an adenovirus construct of the present invention with the biological sample; and (c) analyzing reporter gene activity to detect circulating tumor cells in the biological sample.

In other embodiments, the methods can further comprise contacting the biological sample with a second adenovirus. The second adenovirus construct can infect a different cell type than the first adenovirus construct.

In a specific embodiment, a method for detecting circulating tumor cells in a biological sample from a patient comprises the steps of (a) obtaining a biological sample from a patient, wherein the biological sample comprises a mixed cell population suspected of containing circulating tumor cells; (b) contacting the biological sample with a mixture of adenoviral constructs of the present invention; and (c) analyzing reporter gene activity to detect circulating tumor cells in the biological sample.

In another aspect, the present invention provides methods for detecting a specific cell type or a target cell in a biological sample using the adenoviruses described herein. In one embodiment, a method comprises (a) contacting the adenovirus construct with a biological sample obtained from a patient; and (b) analyzing reporter gene activity to detect specific cell types or target cells in the biological sample. The specific cell type of target cell can include, but is not limited to, a cancer cell, a stromal cell, a mesenchymal cell, an endothelial cell, a fetal cell, a stern cell, and a non-hematopoietic cell.

In the methods described herein, the biological sample can be selected from the group consisting of whole blood, plasma, serum, urine, synovial fluid, saliva, tissue biopsy, surgical specimen, semen, and lavage.

### BRIEF DESCRIPTION OF THE FIGURES

FIG. 1 illustrates the development of adenoviral vectors useful for the ex vivo detection and quantification of viable circulating tumor cells. Conditionally replicative adenoviral vectors are made tissue-specific by placing the E1A gene under the control of the PSA-PBN prostate promoter and enhancer. Human blood is gradient partitioned to remove red blood cells (RBC) and isolate CTCs and mononuclear cells (Buffy Coat). These cells are transiently grown in tissue culture media and infected with CRAD Major Late Transcriptional Unit (MLTU) reporter vectors. With tissue-selective viral replication, the MLTU is activated to produce capsid proteins and secreted reporters (chorionic Gonadotropin, alpha fetal protein, and Metridia Luciferase). Viral replication amplifies viral genome copy and therefore reporter signal (up to 10,000 copies/cell). Secreted CTC-specific reporters from the growth media are quantified by standard assays.

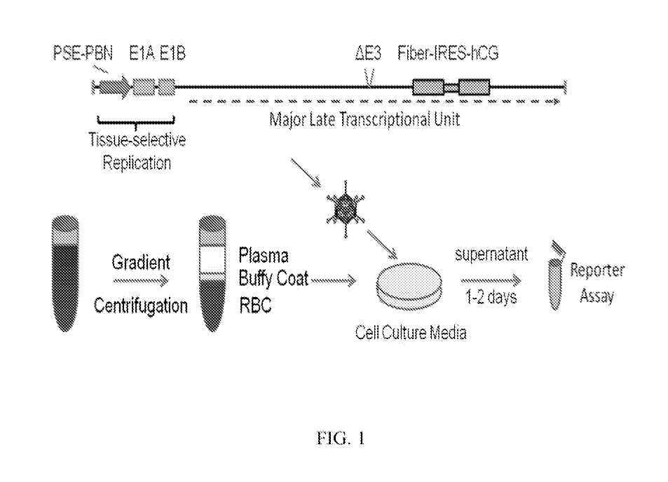

FIG. 2 shows results of tissue-selective replication reporters. The androgen dependent conditionally replicative adenovirus, Ad5PSE-PBN-E1A-AR, was co-infected with the Fiber-1RES-GFP replication reporter FFIG. Androgen (R1881) induced replication of Ad5PSE-PBN-E1A-AR, by evidence of GFP induction, only in the androgen receptor (AR) positive prostate cancer cell line, LNCaP. There is no replication in LNCaP in the absence of R1881. Two AR negative cell lines are included as negative controls. GFP correlated with viral output and capsid protein level.

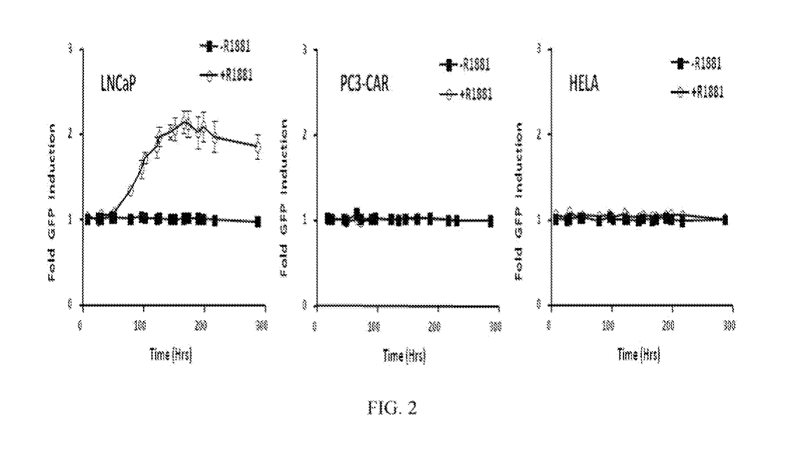

FIG. 3 shows the results of prostate-selective imaging reporters and Fiber-linked reporter expression. The PSE-PBN promoter/enhancer drives E1A and prostate-selective replication of Ad-PSA-Fib. Ad-PSA-Fib-HSVTK is an identical virus with a Fiber-1RES-HSVTK reporter cassette. The control virus Ad-Cntl-Fib lacks PSE-PBN-E1A and is therefore non-replicating. Western blotting shows the correlative expression of Fiber and HSVTK in Ad-PSA-Fib-HSVTK.

FIG. 4 present the results of preliminary studies on partitioning and infection. In FIG. 4A, LNCaP-MLuc stable transfectants were diluted in 10 mls of human blood and the blood was partitioned by ficol gradient centrifugation. Total DNA from the buffy coat was isolated and MLuc DNA was quantified by real time PCR. As few as 1 cell/ml of blood was detectable. In FIG. 4B, LNCaP-MLuc cells were serially diluted in 10⁶ leukemic cells and infected with a fixed amount of Ad5-PSE-PBN-E1A for 2 hours. Total DNA was isolated and recombinant adenovirus was quantified by virus-specific quantitative PCR for the Fiber gene.

FIG. 5 shows the results from the CTC-RV Pilot Assay. LNCaP cells were serially diluted into one million HL60 promyolcytic leukemia cells and infected with AdPSE-PBN-Fiber-1RES-MLuc. MLuc activity was quantified 6 days post-infection. As few as one LNCaP cell in one million HL60 cells was detectable. Error bars=SD.

FIG. 6 shows the results from a patient study. Blood from a single patient with metastatic prostate cancer (under treatment) and a healthy donor were partitioned into multiple aliquots (3 ml) and assayed for CTC signal by AdPSE-PBN-Fiber-1RES-MLuc infection. hMLuc activity was determined 6 days post infection. 500 LNCaP cells were spiked into patient sample as a reference control. Error bards=SD.

### DETAILED DESCRIPTION OF THE INVENTION

It is understood that the present invention is not limited to the particular methods and components, etc., described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to an “adenovirus” is a reference to one or more adenoviruses, and includes equivalents thereof known to those skilled in the art and so forth.

Unless defined otherwise, all technical and scientific terms used herein have the same meaning as commonly understood by one of ordinary skill in the art to which this invention belongs. Specific methods, devices, and materials are described, although any methods and materials similar or equivalent to those described herein can be used in the practice or testing of the present invention.

All publications cited herein are hereby incorporated by reference including all journal articles, books, manuals, published patent applications, and issued patents. In addition, the meaning of certain terms and phrases employed in the specification, examples, and appended claims are provided. The definitions are not meant to be limiting in nature and serve to provide a clearer understanding of certain aspects of the present invention.

### I. Definitions

The term “adenovirus” refers to the virus itself or derivatives thereof. The term covers all serotypes and subtypes and both naturally occurring and recombinant forms, except where otherwise indicated. Thus, the term “adenovirus” or “adenoviral particle” is used to include any and all viruses that can be categorized as an adenovirus, including any adenovirus that infects a human or an animal, including all groups, subgroups, and serotypes. There are at least 51 serotypes of adenovirus that are classified into several subgroups. For example, subgroup A includes adenovirus serotypes 12, 18, and 31. Subgroup C includes adenovirus serotypes 1, 2, 5, and 6. Subgroup D includes adenovirus serotype 8, 9, 10, 13, 15, 1 7, 19, 20, 22-30, 32, 33, 36-39, and 42-49. Subgroup E includes adenovirus serotype 4. Subgroup F includes adenovirus serotypes 40 and 41. These latter two serotypes have a long and a short Fiber protein. Thus, as used herein an “adenovirus” or “adenovirus particle” may include a packaged vector or genome. Depending upon the context, the term “adenovirus” can also include adenoviral vectors.

An “adenovirus vector,” “adenoviral vector,” or “adenovirus construct” is a term well understood in the art and generally comprises a polynucleotide comprising all or a portion of an adenovirus genome. Thus, an “adenovirus vector,” “adenoviral vector,” or “adenovirus construct” refers to any of several forms including, but not limited to, DNA, DNA encapsulated in an adenovirus coat, DNA packaged in another viral or viral-like form (such as herpes simplex, and AAV), DNA encapsulated in liposomes, DNA complexed with polylysine, complexed with synthetic polycationic molecules, conjugated with transferrin, and complexed with compounds such as PEG to immunologically “mask” the molecule and/or increase half-life, and conjugated to a nonviral protein.

In particular embodiments, the adenoviral vector typically contains most of the adenoviral genome. The adenoviral vector may also contain a bacterial origin of replication. In other embodiments, portions of the wild-type adenoviral genome may be deleted to permit insertion of desired products and the packaging of recombinant adenoviral vectors containing the desired genes. In certain embodiments, adenovirus vectors are replication-competent in a target cell. In other embodiments, adenovirus constructs are conditionally replicative in a target cell.

Recombinant adenoviruses are currently used for a variety of purposes, including gene transfer in vitro, vaccination in vivo, and gene therapy. Several features of adenovirus biology have made such viruses the vectors of choice for certain of these applications. For example, adenoviruses transfer genes to a broad spectrum of cell types, and gene transfer is not dependent on active cell division. Additionally, high titers of virus and high levels of transgene expression can generally be obtained.

Decades of study of adenovirus biology have resulted in a detailed picture of the viral life cycle and the functions of the majority of viral proteins. The genome of the most commonly used human adenovirus (serotype 5) consists of a linear, 36 kb, double-stranded DNA molecule. Both strands are transcribed and nearly all transcripts are heavily spliced. Viral transcription units are conventionally referred to as early (E1, E2, E3 and E4) and late, depending on their temporal expression relative to the onset of viral DNA replication. The high density and complexity of the viral transcription units poses problems for recombinant manipulation, which is therefore usually restricted to specific regions, particularly E1, E2A, E3, and E4. In most recombinant vectors, transgenes are introduced in place of E1 or E3, the former supplied exogenously. The E1 deletion renders the viruses defective for replication and incapable of producing infectious viral particles in target cells; the E3 region encodes proteins involved in evading host immunity, and is dispensable for viral production per se.

Two approaches have traditionally been used to generate recombinant adenoviruses. The first involves direct ligation of DNA fragments of the adenoviral genome to restriction endonuclease fragments containing a transgene. The low efficiency of large fragment ligations and the scarcity of unique restriction sites have made this approach technically challenging. The second and more widely used method involves homologous recombination in mammalian cells capable of complementing defective adenoviruses (“packaging lines”). Homologous recombination results in a defective adenovirus which can replicate in the packaging line (e.g., 293 or 911 cells) which supplies the missing gene products (e.g., E1). The desired recombinants are identified by screening individual plaques generated in a lawn of packaging cells. The low efficiency of homologous recombination, the need for repeated rounds of plaque purification, and the long times required for completion of the viral production process have hampered more widespread use of adenoviral vector technology.

As used herein, the term “administration” refers to the act of giving a drug, prodrug, or other agent, therapeutic treatment, or viral construct to a subject (e.g., a subject or in vivo, in vitro, or ex vivo cells, tissues, and organs). Exemplary routes of administration to the human body can be through the eyes (ophthalmic), mouth (oral), skin (transdermal), nose (nasal), lungs (inhalant), oral mucosa (buccal), ear, by injection (e.g., intravenously, subcutaneously, intratumorally, intraperitoneally, etc.) and the like.

The terms “cancer” and “cancerous” refer to or describe the physiological condition in mammals that is typically characterized by unregulated cell growth. A “tumor” comprises one or more cancerous cells. Examples of cancer include, but are not limited to, carcinoma, lymphoma, blastoma, sarcoma, and leukemia or lymphoid malignancies. More particular examples of such cancers include small intestine cancer, bladder cancer, lung cancer, thyroid cancer, uterine cancer, liver cancer, kidney cancer, breast cancer, stomach cancer, testicular cancer, cervical cancer, esophageal cancer, ovarian cancer, colon cancer, melanoma, prostate cancer, and the like. As used herein, the term “cancer cells” refers to individual cells of a cancer.

“Detecting” refers to determining the presence, absence, or amount of a particular cell or target cell in a biological sample. The term specifically includes quantifying the amount of the cell in a sample. For example, the methods and compositions of the present invention can be used to identify whether a biological sample contains a circulating tumor cell, more specifically, whether the cell is viable, as well as identifying the tissue of origin, and the like.

The term “expression” as used herein refers to the transcription and stable accumulation of sense (mRNA) or anti sense RNA derived from the nucleic acid fragment of the invention. Expression may also refer to translation of mRNA into a polypeptide.

The term “operably linked” refers to the association of nucleic acid sequences on a single nucleic acid fragment so that the function of one is affected by the other. For example, a promoter is operably linked with a coding sequence when it is capable of affecting the expression of that coding sequence (i.e., that the coding sequence is under the transcriptional control of the promoter). Coding sequences can be operably linked to regulatory sequences in sense or antisense orientation. In certain embodithents, the term “operably linked” can refer to the association of an enhancer with a promoter in which the enhancer stimulates or enhances promoter activity.

The term “polynucleotide” or “nucleic acid” refers to a polymeric form of nucleotides of any length, either ribonucleotides and/or deoxyribonucleotides. These terms include a single-, double- or triple-stranded DNA, genomic DNA, cDNA, RNA, DNA-RNA hybrid, or a polymer comprising purine and pyrimidine bases, or other natural, chemically, biochemically modified, non-natural or derivatized nucleotide bases. The backbone of the polynucleotide can comprise sugars and phosphate groups (as may typically be found in RNA or DNA), or modified or substituted sugar or phosphate groups. Alternatively, the backbone of the polynucleotide can comprise a polymer of synthetic subunits such as phosphoramidates and thus can be an oligodeoxynucleoside phosphoramidate (P—NH₂) or a mixed phosphoramidate-phosphodiester oligomer. In addition, a double-stranded polynucleotide can be obtained from the single stranded polynucleotide product of chemical synthesis either by synthesizing the complementary strand and annealing the strands under appropriate conditions, or by synthesizing the complementary strand de novo using a DNA polymerase with an appropriate primer.

The following are non-limiting examples of polynucleotides: a gene or gene fragment, exons, introns, mRNA. tRNA, rRNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs, uracyl, other sugars and linking groups such as fluororibose and thioate, and nucleotide branches. The sequence of nucleotides may be interrupted by non-nucleotide components. A polynucleotide may be further modified after polymerization, such as by conjugation with a labeling component. Other types of modifications included in this definition are caps, substitution of one or more of the naturally occurring nucleotides with an analog, and introduction of means for attaching the polynucleotide to proteins, metal ions, labeling compbnents, other polynucleotides, or a solid support.

The term “plasmid” refers to an extrachromosomal circular DNA capable of autonomous replication in a given cell. The range of suitable plasmids is very large. In certain embodiments, the plasmid is designed for amplification in bacteria and for expression in a eukaryotic target cell. Such plasmids can be purchased from a variety of manufacturers. Exemplary plasmids include but are not limited to those derived from pBR322 (Gibco BRL), pUC (Gibco BRL), pBluescript (Stratagene), pREP4, pCEP4 (Invitrogen), pC1 (Promega) and p Poly (Lathe et al., Gene 57 (1987), 193-201). Plasmids can also be engineered by standard molecular biology techniques (Sambrook et al., Laboratory Manual, Cold Spring Harbor Laboratory Press, Cold Spring Harbor (1989), N.Y.). It may also comprise a selection gene in order to select or to identify the transfected cells (e.g., by complementation of a cell auxotrophy or by antibiotic resistance), stabilizing elements (e.g., cer sequence) or integrative elements (e.g., LTR viral sequences and transposons).

The term “shuttle plasmid” refers to a plasmid comprising a unique restriction site between certain homologous recombination sites and used to insert a desired nucleic acid molecule, i.e., a. nucleic acid molecule encoding a desired product, into a recombinant adenoviral vector. The homologous recombination sites can be, for example. Ad5 right and Ad5 left. In further embodiments, the shuttle plasmid may have a tissue specific promoter which controls the expression of the desired nucleic acid molecule. The shuttle plasmid also contains a majority of the viral genes necessary to form viral particles. However, the shuttle plasmid does not contain all necessary genes to form viral particles.

The term “polypeptide” or “peptide” refers to a polymeric form of amino acids of any length, which may include translated, untranslated, chemically modified, biochemically modified, and derivatized amino acids. A polypeptide or peptide may be naturally occurring, recombinant, or synthetic, or any combination of these. Moreover, the term “polypeptide” or “peptide,” as used herein, refers to proteins, polypeptides, and peptides of any size, structure, or function. For example, a polypeptide or peptide may comprise a string of amino acids held together by peptide bonds. A polypeptide or peptide may alternatively comprise a long chain of amino acids held together by peptide bonds. Moreover, a polypeptide or peptide may also comprise a fragment of a naturally occurring protein or peptide. A polypeptide or peptide may be a single molecule or may be a multi-molecular complex. In addition, such polypeptides may have modified peptide backbones as well. The term “polypeptide” or “peptide” further comprises immunologically tagged proteins and fusion proteins, including, but not limited to, fusion proteins with a heterologous amino acid sequence, fusion proteins with heterologous and homologous leader sequences, and fusion proteins with or without N-terminal methionine residues.

“Promoter” refers to a DNA sequence capable of controlling the expression of a coding sequence or functional RNA. In general, a coding sequence is located 3′ to a promoter sequence. In some embodiments, the promoter sequence comprises proximal and more distal upstream elements, the latter elements often referred to as enhancers. Accordingly, an “enhancer” is a DNA sequence which can stimulate promoter activity and may be an innate element of the promoter or a heterologous element inserted to enhance the level or tissue-specificity of a promoter. Promoters may be derived in their entirety from a native gene, or may comprise different elements derived from different promoters found in nature, or even comprise synthetic DNA segments. It is understood by those skilled in the art that different promoters may direct the expression of a gene in different tissues or cell types, or at different stages of development, or in response to different environmental conditions. It is further recognized that since in most cases the exact boundaries of regulatory sequences have not been completely defined, DNA fragments of different lengths may have identical promoter activity.

The term “replication” means duplication of a vector. This duplication, in the case of viruses, can occur at the level of nucleic acid, or at the level of infectious viral particle. In the case of DNA viruses, replication at the nucleic acid level comprises DNA replication. In the case of RNA viruses, nucleic acid replication comprises replication into plus or minus strand (or both). In the case of retroviruses, replication at the nucleic acid level includes the production of cDNA as well as the further production of RNA viral genomes. The essential feature is the generation of nucleic acid copies of the original viral vector. However, replication also includes the formation of infectious DNA or RNA viral particles. Such particles may successively infect cells in a given target tissue, thus distributing the vector through all or a significant portion of the target tissue.

The terms “sample,” “biological sample,” “patient sample” and the like, encompass a variety of sample types obtained from an individual, subject or a patient and can be used in a diagnostic or monitoring assay. Moreover, a sample obtained from a patient can be divided and only a portion may be used to for diagnosis. Further, the sample, or a portion thereof, can be stored under conditions to maintain sample for later analysis. The definition specifically encompasses blood and other liquid samples of biological origin (including, but not limited to, serum, plasma, urine, saliva, stool and synovial fluid), solid tissue samples such as a biopsy specimen or tissue cultures or cells derived therefrom and the progeny thereof. The definition also includes samples that have been manipulated in any way after their procurement, such as by centrifugation, filtration, precipitation, dialysis, chromatography, treatment with reagents, washed, or enriched for certain cell populations including tumor cells and the like. The terms further encompass a clinical sample, and also include cells in culture, cell supernatants, tissue samples, organs, bone marrow, and the like. In a specific embodiment, a sample comprises a blood sample. In another embodiment, a serum sample is used.

As used herein, the term “subject” refers to any animal (e.g., a mammal), including, but not limited to, humans, non-human primates, rodents, and the like (e.g., which is to be the recipient of a particular treatment). Typically, the terms “subject” and “patient” are used interchangeably, unless indicated otherwise herein.

As used herein, the terms “treatment,” “treating,” “treat” and the like, refer to obtaining a desired pharmacologic and/or physiologic effect. The terms are also used in the context of the administration of a “therapeutically effective amount” of an agent, e.g., a viral construct of the present invention. The effect may be prophylactic in terms of completely or partially preventing a particular outcome, disease or symptom thereof and/or may be therapeutic in terms of a partial or complete cure for a disease and/or adverse affect attributable to the disease. “Treatment,” as used herein, covers any treatment of a disease or condition in a subject, particularly in a human, and includes: (a) preventing the disease or condition from occurring in a subject which may be predisposed to the disease or condition but has not yet been diagnosed as having it; (b) inhibiting the disease or condition, i.e., arresting its development; and (c) relieving the disease or condition, e.g., causing regression of the disease or condition, e.g., to completely or partially remove symptoms of the disease or condition. In particular embodiments, the term is used in the context of treating a subject with cancer.

As used herein, the term “vector” refers to a polynucleotide construct designed for transduction/transfection of one or more cell types. Vectors may be, for example, “cloning vectors” which are designed for isolation, propagation and replication of inserted nucleotides, “expression vectors” which are designed for expression of a nucleotide sequence in a host cell, or a “viral vector” which is designed to result in the production of a recombinant virus or virus-like particle, or “shuttle vectors,” which comprise the attributes of more than one type of vector.

In one aspect, the present invention provides virus constructs/vectors useful for detecting specific cell types in a biological sample. In particular embodiments, the present invention utilizes tissue-specific (also referred to as cell type specific) conditionally replicating adenoviruses (CRADs). In a specific embodiment, the present invention provides prostate-specific CRADs utilizing secreted MLTU reporter genes for the ex vivo detection and quantification of viable disseminated prostate cancer (PCa) cells.

The use of reporter vectors (e.g., adenovirus reporter vectors) to detect Circulating Tumor Cells (CTCs) (also referred to as disseminated tumor cells) in a biological sample provides numerous advantages to present techniques. Epithelial cell partitioning may not be necessary because adenoviruses naturally infect epithelial cells while not infecting leukocytes. Leon et al., 95(22) PROC. NATL. ACAD. SCI. U.S.A. 13159-64 (1998). Tissue-specific/cell specific promoters, such as probasin, allow for identification of tumor cells of a specific tissue origin. CTC signals should be cancer-specific because detached normal epithelial cells die by anoikis. Ciarugi and Giannoni, 76(11) BIOCHEM. PPHARMACOL. 1352-64 (2008). Accordingly, only viable cells should be susceptible to infection and reporter gene expression. Viral replication provides a mechanism for specificity and up to about 10,000-fold signal enrichment. Finally, the use of secreted reporters in certain embodiments separates the CTC signal from the overwhelming background of mononuclear cells, proteins, debris, and nucleic acids. The use of reporter genes which have established, high sensitivity and certified assays, such as hCG and AFP, is intended to make this assay widely accessible and to overcome the need for specialized knowledge or analysis equipment. Most importantly, the impact of this technology is broad because it is easily adaptable to other tumors and other cell types by simply using alternate tissue-selective/cell specific promoter and enhancer cassettes.

Accordingly, in one embodiment, an adenovirus construct comprises (a) a cell type specific promoter that drives adenoviral replication; and (b) at least one reporter gene incorporated into the viral Major Late Transcriptional Unit. In another embodiment, an adenovirus construct comprises a cell type specific promoter that drives adenoviral replication. In such a case, the amplified viral genome itself can be utilized the detect and quantify the level of viable target cells (e.g., prostate tumor cells) per volume of blood, serum or prostatic fluid. The level of viable CTCs may correlate with disease burden and thus, may be predictive of outcome. The adenovirus construct can further comprise an enhancer operably linked to the cell type specific promoter. In one embodiment, the cell type specific promoter is operably linked to the E1 gene. In other embodiments, the cell type can be selected from the group consisting of a cancer cell, a stromal cell, a mesenchymal cell, an endothelial cell, a fetal cell, a stem cell, and a non-hematopoietic cell. Indeed, reporter gene(s) can be used to “tag” a particular cell type for partitioning from other non-disseminated cells. For example, a recombinant reporter virus can be used to partition fetal cells from maternal bodily fluids so that chromosome copy number or genetic rearrangement can be quantified in the absence of contaminating maternal genome. Conditional replication of the reporter virus in fetal cells may be necessary for efficient detection and partitioning of the fetal cells.

In a specific embodiment, the cell type is a cancer cell. The type of cancer includes, but is not limited to, small intestine cancer, bladder cancer, lung cancer, thyroid cancer, uterine cancer, liver cancer, kidney cancer, breast cancer, stomach cancer, testicular cancer, cervical cancer, esophageal cancer, ovarian cancer, colon cancer, melanoma, prostate cancer, and the like.

In a further embodiment, the reporter gene is a secreted reporter. The secreted reporter gene can include, but is not limited to, human chorionicgonadotrophin (hCG), alpha fetal protein (AFP), humanized Metridia luciferase (hMLuc), Gaussia Luciferase, Cypridina Luciferase, Secreted Alkaline Phosphatase, and the like.

The present invention also provides an adenovirus construct comprising (a) a prostate cancer cell specific promoter that drives adenoviral replication; and (b) at least one reporter gene incorporated into the viral Major Late Transcriptional Unit. In an alternative embodiment, an adenovirus construct comprises a prostate cancer cell specific promoter that drives adenoviral replication. In such a case, the amplified viral genome itself can be utilized the detect and quantify the level of viable circulating prostate tumor cells per volume of blood, serum or prostatic fluid. The prostate cancer cell specific promoter may comprise prostate selective probasin promoter. In another embodiment, the prostate cancer cell specific promoter is operably linked to the E1 gene. In other embodiments, the promoter can be operably linked to the E1A, E1B, E2, E3 and/or E4 genes.

Other prostate cancer cell specific promoters can be used including, but not limited to, Prostate Specific Antigen promoter, Probasin promoter, Prostate Specific Membrane Antigen promoter, Prostate Stem Cell Antigen promoter, Semenogelin promoter, KLK4 promoter, NKX3.1 promoter, AMACAR promoter, Uroplakin II promoter, Uroplakin Ia, Ib, II, and III, Desmin promoter, Elastase-1 promoter, Endoglin promoter, Flit-1 promoter, GFAP promoter, ICAM-2 promoter, INF-alpha promoter, INF-beta promoter, OG-2 promoter, SP-B promoter, Syn1 promoter, Albumin promoter, AFP promoter, CCKAR promoter, CEA promoter, c-erb2 promoter, COX-2 promoter, CXCR4 promoter, E2F-1 promoter, LP promoter, MUC1 promoter, Survivin promoter, TRP1 promoter, Tyr promoter, Uromodulin promoter, PCK1 promoter, CHDH promoter, ASPA promoter, PKLR promoter, TCF2 promoter, PKHD1 promoter, UPB1 promoter, SSTR1 promoter, HYAL1 promoter, FANCA1 promoter, KLRC3 promoter, KLRC2 promoter, APOBEC1 promoter, CEACAM1 promoter, GYS2 promoter, ADH4 promoter, ALB promoter, SFTPB promoter, PLUNC promoter, WISP2 promoter, PRLR promoter, WT1 promoter, PAEP promoter, FOLR1 promoter, V1T promoter, UCN3 promoter, IPF1 promoter, 1 NS promoter, CTRB1 promoter, S1 promoter, MAGEA4 promoter, Telomerase promoter, and the like.

In certain embodiments, the adenovirus construct can further comprise an enhancer operably linked to the prostate cancer cell specific promoter. In a specific embodiment, the enhancer comprises prostate specific antigen enhancer. Other prostate cancer cell specific enhancers can be used including, but not limited to, Prostate Specific Antigen enhancer, Prostate Specific Membrane Antigen enhancer, Probasin Enhancer, and Prostate Stem Cell Antigen Enhancer.

The reporter is a secreted reporter in some embodiments, and can include hCG, AFP, hMLuc, Gaussia Luciferase, Cypridina Luciferase, and Secreted Alkaline Phosphatase. In a specific embodiment, the at least one secreted reporter gene expresses hCG, AFP, and/or hM Luc.

In a more specific embodiment, the present invention provides an adenovirus construct comprising (a) prostate selective probasin promoter operably linked to the E1 gene; and (b) prostate specific antigen enhancer operably linked to the probasin promoter. The adenovirus construct can further comprise at least one reporter gene incorporated into the viral Major Late Transcriptional Unit. In particular embodiments, the reporter gene is a secreted reporter. The secreted reporter gene can be selected from the group consisting of hCG, AFP, hMLuc, Gaussia Luciferase, Cypridina Luciferase, Secreted Alkaline Phosphatase, and the like.

In another embodiment, the present invention provides an adenovirus construct comprising a cell-type specific promoter operably linked to a reporter gene. The reporter gene can be inserted into any of the five early (E1A, E1B, E2, E3 and E4), four intermediate (1Va2, 1X, VAI, and VAII), or the Major Late Transcrptional Unit. In a particular embodiment, the reporter gene is inserted into the E1 gene. The present invention also provides a kit comprising such an adenovirus construct and a helper virus. This type of two virus system can be used to detect disseminated cells in biological samples. The co-administered helper or replicating virus complements the replication of the reporter virus. The present invention also provides methods for detecting circulating tumor cells in a biological sample from a patient. In one embodiment, the method comprises the steps of (a) contacting an adenovirus construct with the biological sample obtained from a patient; and analyzing reporter gene activity to detect circulating tumor cells in the biological sample. In another embodiment, the method can comprise the steps of (a) obtaining a biological sample from a patient, wherein the biological sample comprises a mixed cell population suspected of containing circulating tumor cells; (c) contacting an adenovirus construct with the biological sample; and analyzing reporter gene activity to detect circulating tumor cells in the biological sample. The methods may further comprise contacting the biological sample with a second adenovirus construct of the present invention. In particular embodiments, the second adenovirus construct infects a different cell type than the first adenovirus construct. Indeed, multiple viruses that target different cell types can be used in the same biological sample, for example, a mixture of kidney cancer, bladder cancer and prostate cancer reporter viruses.

In yet another embodiment, a method for detecting circulating tumor cells in a biological sample from a patient comprises the steps of (a) obtaining a biological sample from a patient, wherein the biological sample comprises a mixed cell population suspected of containing circulating tumor cells; (c) contacting the biological sample with a mixture of adenoviral constructs; and analyzing reporter gene activity to detect circulating tumor cells in the biological sample.

The present invention also provides methods for detecting specific cell types (or target cells) in a biological sample. In one embodiment, a method for detecting a specific cell type in a biological sample from a patient comprises the steps of (a) contacting an adenovirus construct with a biological sample obtained from a patient; and (b) analyzing reporter gene activity to detect the specific cell type in the biological sample. The biological samples described herein can include, but are not limited to, whole blood, plasma, serum, urine, synovial fluid, saliva, tissue biopsy, surgical specimen, semen, and lavage.

In another aspect, the present invention provides virus constructs/vectors useful for detecting specific cell types in a biological sample. In certain embodiments, the virus used is an adenovirus. In another embodiment, the virus is a retrovirus. Other viruses can be used in the context of the present invention including, but not limited to, herpes simplex virus, influenza virus, Newcastle disease virus, poliovirus, reovirus, vaccinia virus and vesicular virus.

In another aspect, the present invention provides pharmaceutical compositions comprising a viral construct as described herein. In particular embodiment, the present invention provides pharmaceutical compositions comprising an adenovirus construct.

Without further elaboration, it is believed that one skilled in the art, using the preceding description, can utilize the present invention to the fullest extent. The following examples are illustrative only, and not limiting of the remainder of the disclosure in any way whatsoever.

### EXAMPLES

The following examples are put forth so as to provide those of ordinary skill in the art with a complete disclosure and description of how the compounds, compositions, articles, devices, and/or methods described and claimed herein are made and evaluated, and are intended to be purely illustrative and are not intended to limit the scope of what the inventors regard as their invention. Efforts have been made to ensure accuracy with respect to numbers (e.g., amounts, temperature, etc.) but some errors and deviations should be accounted for herein. Unless indicated otherwise, parts are parts by weight, temperature is in degrees Celsius or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described processes. Only reasonable and routine experimentation will be required to optimize such process conditions.

### Example 1

### Development of Prostate-Selective Circulating Tumor Cell Reporter Vectors (CTC-RV)

CTC-RV specificity is achieved by prostate-selective viral replication. The early viral E1A gene, which is necessary for viral replication, is placed under the control of the prostate-selective probasin promoter and Prostate Specific Antigen enhancer (PSE-PBN). Three secreted reporter genes (HCG, AFP, and hMLuc) are independently incorporated into the viral Major Late Transcriptional Unit (MLTU). Thus, viral replication and reporter gene expression are limited to PCa cells. Specificity is further achieved by the inherent poor adenoviral infection rate of hematologic cells. Quantitative milestones are described in the section below.

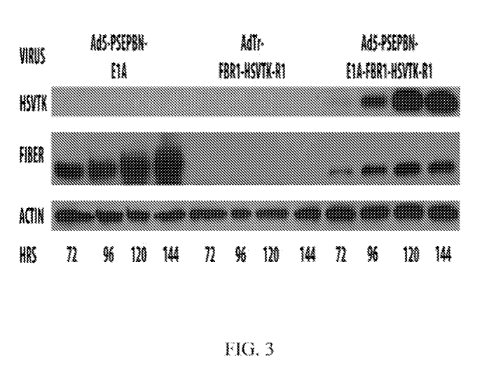

A prostate-selective CRAD vector is being developed which expresses the PET imaging reporter, HSVTK, via a Fiber-1RES (Internal Ribosome Entry Site) cassette (Ad-PSA-Fib-HSVTK). FIG. 3 demonstrates that HSVTK reporter gene expression is concurrent with viral replication and Fiber capsid protein expression. The HSVTK gene is functional and results in specific uptake of the HSVTK substrate ³H-GCV (gancyclovir) (data not shown). Non-replicative control viruses lacking the transgene (Ad-Cntr1-Fib) or lacking the prostate-specific replication cassette (Ad-Cntr1-Fib-HSVTK) do not express HSVTK (FIG. 3). Ad-PSA-Fib-HSVTK replication is prostate-selective and the Fiber-1RES-HSVTK has no negative effects on viral replication or production (data not shown).

Using prostate-selective CRADs and non-invasive viral replication reporters, prostate-selective replicating adenoviruses which express secreted reporters through Fiber-1RES cassettes are developed. Specifically, three reporters are linked to adenoviral fiber gene expression: Human chorionic gonadotropin (hCG), Alpha Fetal Protein (AFP), and a novel humanized Metridia Luciferase reporter (hMLuc).

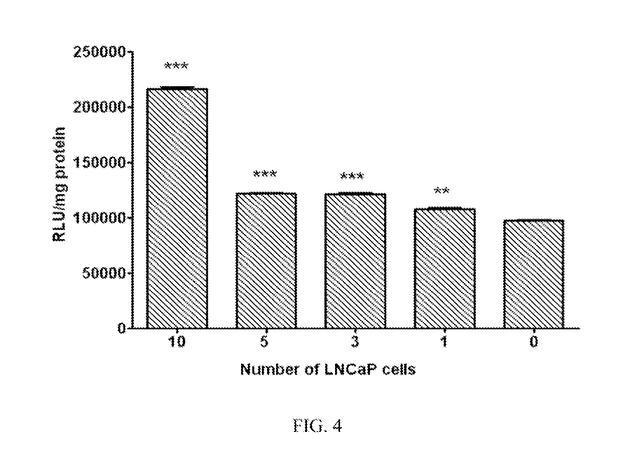

Prostate-Selective CTC-RVs. An E3-deleted serotype 5 adenoviral vector, pPSE-PBN-E1A-fex, contains a prostate-selective replication cassette in the E1 region of the pFex viral vector. This parental vector contains all of the necessary components to generate an active virus, minus the Fiber gene, which has been replaced by the negative selectable gene, SacB, surrounded by modified lox sites. As previously described (Lupold et al., 35(20) Nucleic ACIDS RES. e138 (2007)), cre recombinase can uni-directionally transfer modified Fiber gene cassettes from Rpuc-Fib-shuttle vectors directly into the natural Fiber gene locus, thus replacing SacB. This exact vector and strategy was used to create the HSVTK reporter viruses in FIG. 4. Three RPuc-Fiber-1RES-reporter shuttle vectors: RPuc-Fib-1RES-MLuc, RPuc-Fib-1RES-hCG, and RPuc-Fib-1RES-AFP are generated. These three plasmids are recombined with the pPSE-PBN-E1A-Fex viral genome to create the desired CTC-RVs. Adenovirus is amplified in DPL-S11 cells, a derivative of PER.C6 which is designed to eliminate the development of Replication Competent Adenovirus (a rare event where adenovirus revert to wild type E1 region via homologous recombination with complementing adenoviral early regions present in the packaging cell line's genome). Fallaux et al., (13) HUM. GENE THER. 1909-17 (1998). Virus is purified by Cesium Chloride density gradient centrifugation or commercially available column kits and titered by hexon immunohistochemical methods as previously described. See Ribas et al., 69(18) CANCER RES. 7165-69 (2009); Hoti et al., 15(8) MOL. THER. 1495-1503 (2007); Lupold et al., 35(20) NUCLEIC ACIDS RES. e138 (2007); Hoti et al., 14(6) MOL. THER. 768-78 (2006); Li et al., 62(9) CANCER 121:8. 2576-82 (2002); DeWeese et al., 61(20) CANCER RES. 7464-72 (2001); and Rodriguez et al., 57(13) CANCER RES. 2559-63 (1997).

In addition to these CRAD vectors, non-replicating versions of each adenovirus (Ad-CMV-Reporter) are generated as positive controls for reporter gene expression and assay development. In summary, Example 1 results in the following deliverables: 3 replicating CTC-RV, 3 non-replicating adenovirus and two control viruses. A quantitative milestone of each adenovirus at concentrations≧10¹⁰ infectious units (IU)/ml is achieved.

Reporter Assays. Particular embodiments of the present invention utilize a humanized version of a secreted luciferase from the marine copepod Metridia longa⁵⁸. Humanized Metridia luciferase (hMLuc) activity corresponded linearly with cell number in prostate cancer cell models over 4-log dynamic range (data not shown). In addition to hMLuc, two additional secreted reporters, hCG and AFP, are used. hCG is a secreted glycoprotein hormone which is detectable at low levels in the serum of pregnant women or patients with trophoblastic tumors, choriocarcinoma, and testicular tumors. See McPherson et al., HENRY'S CLINICAL DIAGNOSIS & MANAGEMENT BY LABORATORY METHODS 21^{st} Ed., Philadelphia: Sauders Elsevier (2007). Commercially available serum assays for hCG are readily used in the clinic and laboratory with sub-picomolar sensitivity. The NovaTec hCG ELISA kit, with reference standard 1-400 mIU/ml and sensitivity to 0.5 IU/ml, is used. AFP is secreted by embryonic hepatocytes and fetal yolk sac cells in pregnancy and often in patients with hepatocellular carcinoma and germ cell tumors. Id. Commercially available serum assays for AFP are readily used in the clinic and laboratory with sub-picomolar sensitivity. The NovaTec AFP ELISA kit, with reference standard 5-200 ng/ml and 0.1 ng/ml sensitivity, is used. Additional secreted reporters such as alkaline phosphatase can be utilized. Similarly, alternative reporters and high sensitivity assays can also be used.

Non-replicating (Ad-CMV-Reporter) adenovirus is used to infect LNCaP cells at a multiplicity of infection (MOI) ranging from 1-100 to establish working AFP, hCG, and MLuc assays. Media from infected cells is analyzed about 24-72 hours after infection. Internal standards and linear regression analysis are used to determine assay linearity and sensitivity. Non-infected and empty vector virus serve as negative controls. The following quantitative milestones are achieved: (1) identify the most sensitive reporter and assay pair by viral serial dilution (MOI 10¹-10⁴). (2) Calculations, based on cell number and MOI, are utilized to estimate the minimum number of detectable cells by each assay.

CTC-RV Assays. CRAD reporter adenovirus is assayed for prostate-selective replication by co-infecting AR positive and negative cells with each CTC-RV and the FFIG reporter virus. Each individual virus is then used to infect LNCaP, C42, and CWR22 cells over a range of MOI and corresponding reporter activity is quantified. The following quantitative milestones are achieved: (1) Optimal timing for reporter expression. (2) assay linearity, and (3) viral output:input assays to determine the number of viral particles produced per cell.

### Example 2

### Establishment of Optimal Conditions for CTC Partitioning, Detection and Infection

Efficient detection and quantification of disseminated prostate cancer cells (PCa cells) requires optimized partitioning and infection protocols. Genetically-tagged LNCaP cells are serially diluted and spiked into media or whole blood and various recovery protocols are evaluated. Recovered PCa cells, in the presence of at least about 10⁷ background cells, are infected with serially diluted adenovirus, for various times, to determine the optimal conditions for infection and transgene expression.

The present example is dedicated to the mechanics of tumor cell separation, recovery, infection, and timing. Ten milliliters of blood from normal human donors were spiked with 10-100,000 LNCaP prostate tumor cells stably transfected with hMLuc. Mononuclear cells were isolated in the Buffy coat with BD Vacutainer® CPT™ cell preparation tubes an resuspended in serum supplemented RPMI1640. The mixed cell population was then incubated for 72 hours at 37° C./5% CO₂. Cells were harvested, washed, and total DNA was extracted and subjected to quantitative PCR for the MLuc transgene. FIG. 4A demonstrates that, with this methodology, at least 1 LNCaP cell/ml of blood can be recovered and detected. Linearity of detection is lost with more diluted samples because the LNCaP genome represents only a fraction of the total Buffy coat DNA and is therefore not sampled in every aliquot of DNA for PCR (FIG. 4A).

In a similar study, LNCaP-MLuc cells were serially diluted in one million HL60 leukemic cells and infected with 10⁷ Infectious Units (IU) of Ad5-PSE-PBN-E1 A-AR CRAD. Seventy-two hours later, cells were washed, total DNA was harvested, and viral genome level was quantified by Fiber QPCR. Importantly, viral genomic DNA correlated with PCa cell number (FIG. 4B). Similar trends were found in studies with human blood; however, the limit of detection was much less given the larger number of non-specific cells. Further, the viral genome is only 0.001% the size of the human genome and is therefore rarely sampled in standard QPCR reactions.

These preliminary studies demonstrate that it is feasible to recover CTCs from blood, expand them for days ex vivo, quantify their levels, and infect them with recombinant adenovirus. These early studies represent an initial QPCR approach to quantify disseminated tumor cells by tissue-specific CRAD replication. The development of secreted reporters to separate the signal from the cell and debris background can be used as an alternative approach.

It is estimated that metastatic patients have approximately one CTC for every 10⁵⁻10⁷ blood mononuclear cell. Allan and Keeney, 2010 J. ONCOL. 426218 (2010). Accordingly, at least one CTC is recovered and infected from a background of 10⁷ cells. The target population is Androgen Receptor (AR) positive PCa cells. AR is a justifiable target for CTC quantification as it is expressed in the majority of hormone naïve PCa cells, by evidence of PSA expression, and is further re-activated in the advanced hormone refractory prostate cancers by a variety of mechanisms. See Hu et al., 69(1) CANCER RES. 16-22 (2009); Dehm et al., 68(13) CANCER RES. 5469-77 (2008); Mohler, J. L., 617 ADV. EXP. MED. BIOL. 223-34 (2008); Jagla et al., 148(9) ENDOCRINOLOGY 4334-43 (2007); Sun et al., 25(28) ONCOGENE 3905-13 (2006); Scher and Sawyers, 23(32) J. CLIN. ONCOL. 8253-61 (2005); Ford et al., 170(5) J. UROL. 1817-21 (2003); Linja et al., 61(9) CANCER RES. 3550-55 (2001); Mononen et al., 60(22) CANCER RES. 6479-81 (2000); Kovisto and Rantala, 187(2) J. PATHOL. 237-41 (1999);Taplin et al., 332(21) N. ENG. J. MED. 1393-98 (1995); Visakorpi et al., 9(4) NAT. GENET. 410-06 (1995).

Three separate AR positive PCa cell lines, LNCaP, C4-2, and CWR22, are stably transfected with the GFP expression plasmid, pEGFP-N1. Each GFP-tagged cell line is serially diluted to achieve a ratio of about 1-10,000 tumor cells per 10 mls of blood, which will on average contain ˜5×10⁷ leukocytes. Cells are partitioned by a variety of techniques to identify the optimal partitioning strategy. Specifically, blood is collected in various anti-coagulant tubes (K₂EDTA, EDTA, Heparin) and partitioned by either density gradient centrifugation (to isolate the buffy coat) or by processing with red blood cell lysis protocols and centrifugation. Resulting cell slurries are resuspended in media and analyzed for GFP positive cells by flow cytometry (Guava EasyCyt) or microscopy. Leukocyte labeling may also be applied to quantify total initial cell number. As an alternative to Guava EasyCyt, FACS core facilities are available, as well as fluorescent microscopy or QPCR methods (FIG. 4). Alternatively, stable reporter expressing cells (MLuc, hCG, or AFP) could be used to optimize cell partitioning and recovery. Conditions are optimized to identify the gentlest condition to obtain the maximum percent recovery and repeated to determine the linear regression. The following quantitative milestones are achieved: (1) the ability to recover at least about 75% of input PCa cells, (2) the ability to purify at least about one PCa cell per 10⁷ mononuclear cells, (3) the linear recovery range, and (4) the coefficient of variation.

In addition to effective CTC partitioning, the ability to infect at least about 1 PCa cell line in the background of about 10⁷ non-specific cells is demonstrated. Three AR positive cell lines are serially diluted (1-1,000 cells) into a total of about 10⁸HL60 leukemia cells (as in FIG. 48) and infected (10⁶-10⁹ IU) with the non-replicating GFP-expressing adenovirus, AdTrack. See Luo et al., 2(5) NAT. PROTOC. 1236-47 (2007); and Lupold et al., 35(2) NUCLEIC ACIDS RES. e138 (2007). The total cellular population is harvested about 48-72 hours post infection and analyzed for GFP positive cells by flow cytometry as above (Guava). Infection, growth. and media conditions (volume, type) are also optimized. Reporters such as firefly luciferase or MLuc can be applied in addition to or as an alternative to GFP expression levels or guava sensitivity. The following quantitative milestones are achieved: (1) infection of at least about 1 CTC per 10⁷ non-specific cells and (2) optimal IU/cell ratio, and (3) the linear recovery range.

### Example 3

### CTC Viability

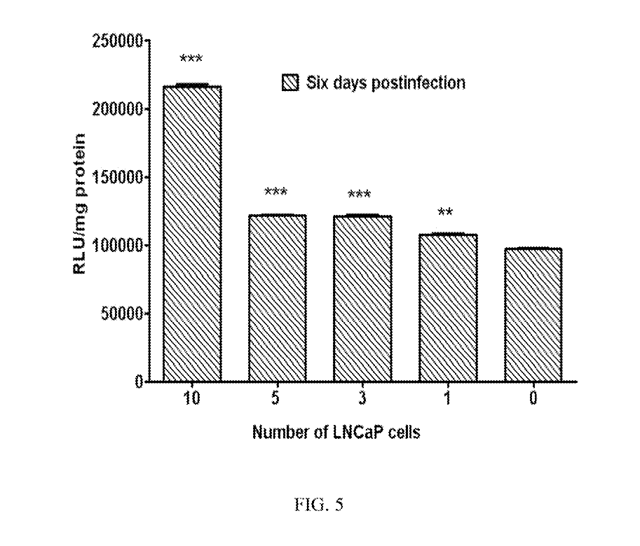

While the literature supports that CTCs from animal and cell line models remain viable for several days (Fong et al., 146(3) SURGERY 498-505 (2009); Kojima et al., 119(10) J. CLIN. INVEST. 3172-81 (2009); Pfitzenmaier et al., 25(3) UROL. ONCOL. 214-20 (2007); and Glinsky et al., 5(2) CELL CYCLE 191-97 (2006)), the true lifespan of a CTC ex vivo remains unknown. However, the inventors have shown that CTCs remain viable for a long enough time to be detected by CTC-RV assays. One CTC-RV has been generated on a pilot scale and purity. More specifically, AdPSE-PBN-Fiber-1RES-MLuc contains a prostate-selective replication cassette and the Fiber-1RES-hMLuc reporter gene. This CTC-RV is capable of detecting as few as one LNCaP cell in one million human promyolocytic leukemia cells (FIG. 5).

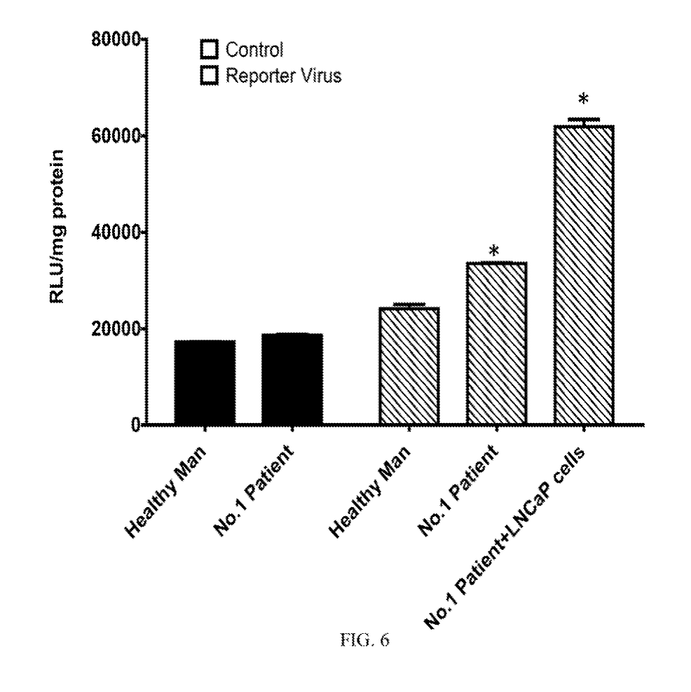

From pilot studies of this CTC-RV with LNCaP cells diluted in de-identified, pooled, and expired blood samples, it was found that partitioning by red blood cell lysis is effective and could detect as few as 10 LNCaP cells per milliliter of blood (data not shown). Importantly, these LNCaP cells remain viable and continue to produce reporter signal for at least nine days post infection. In addition, a single blood sample from a patient with prostate cancer (hormone refractory metastatic disease under treatment) was tested. A reference of 500 LNCaP cells was spiked into the patient sample. In this single study, a significant signal was detectable in the patient's blood, when compared to a healthy volunteer, for as long as six days post infection (FIG. 6). These results support that CTCs will remain viable and detectable over a period of several days.

### Example 4

### Development of Viable Prostate-Specific CTC Quantitative Assay

The optimized CTC-RV, cell partitioning method, infection rate, and time are applied to serially diluted LNCaP, C4-2, and CWRV22 cells in whole blood. About 1 viable PCa CTC/ml of whole blood is detected. The reproducibility of the assay and correlation to CellSearch CTC signal is also determined. A pilot study is performed on a sample set (20 patients/group) of men with newly diagnosed and untreated metastatic PCa, newly diagnosed and untreated local PCa, and men with no known malignancies (control).

The goal of the present example is to combine the technologies and methods of the first two example to achieve a final working assay. Three AR positive PCa cell lines, LNCaP, C42, and CWR22 are serially diluted (1-10,000 cells) per 10 ml of normal human blood. Cells are partitioned by the optimal method determined in Example 2 and re-suspended in serum supplemented media. The heterogeneous cell population is infected with the most sensitive CTC-RV, at pre-determined ranges of MOI, and reporter levels are quantified at pre-determined times (determined in Example 1). These experiments are optimized to achieve the quantitative milestone of detecting a minimum of about 1 CTC/ml of blood. The optimal reporter assay is repeated a minimum of five times to determine assay reproducibility and the quantitative milestone of assay variance. If signal is detectable in fresh blood, the effect of time before processing (1-4 hours after collection) on CTC detection is evaluated.

Patient Sample Trials. The final working assay is evaluated by comparing 3 groups of 20 men each: Group 1: newly diagnosed, untreated metastatic prostate cancer; Group 2: newly diagnosed untreated localized prostate cancer; Group 3: men with no known malignancy (controls). Group 1 is enrolled from among new patients coming to the Johns Hopkins Sidney Kimmel Cancer Center; Groups 2 and 3 are enrolled as part of an ongoing case-control study conducted by Dr. Trock, where the controls are men who are being seen at the Urology clinic for reasons unrelated to cancer. The three groups are matched on age and race and assayed in a blinded fashion (as best achievable). Because previous studies with CellSearch have shown a median of 4 CTCs per 7.5 ml of blood in metastatic prostate cancer patients (Helo et al., 55(4) CLIN. CHEM. 765-73 (2009); and Goodman et al., 18(6) CANCER EPIDEMIOL. BIOMARKERS PREV. 1904-13 (2009), it is anticipated that the assay of the present invention demonstrates the presence of viable and detectable disseminated PCa cells in the majority of patients with advanced disease. In contrast, few patients with localized prostate cancer (Id.) or normal controls (Davis et al., 179 (6) J. UROL. 2187-91 (2008)) have detectable CTCs with CellSearch, nonetheless it is expected that the assay of the present invention demonstrates differences among the groups. The quantitative milestone of CTC cells per patient is determined in each of the 3 groups, and mean CTC counts are compared among the groups using, e.g., analysis of variance (ANOVA) or the nonparametric Kruskal-Wallis test. Because this is a technology development project, the analysis of human samples is not based on a formal power calculation.

Assay Reproducibility and Comparison to CellSearch. Repeat blood samples are obtained to determine assay reproducibility. Alternatively, some blood samples are separated into smaller volumes (and assay scaled down) to determine variability. In addition, at least five CTC-RV positive patients are evaluated by the Veridex CellSearch assay system. Blood samples are obtained and quantified for CTC level and directly compared to CTC-RV. A quantitative milestone of viable cells (as determined by CTC-RV) versus total CTC cells is determined.

In alternative embodiments, fluorometric reporters, multiplexed reporters, alternative promoters, and/or vectors with improved tropism are generated and tested. Furthermore, assay variation/reproducibility and CellSearch comparison can be performed on PCa cell lines diluted in blood.

### Example 5

### Development of Other Prostate-Selective CTC-RVs

Example 1 through 4 above are repeated using different prostate cancer cell specific promoters. The prostate cancer cell specific promoter can include, but is not limited to, Prostate Specific Antigen promoter, Probasin promoter, Prostate Specific Membrane Antigen promoter, Prostate Stem Cell Antigen promoter, Semenogelin promoter, KLK4 promoter, NKX3.1 promoter, AMACAR promoter, Uroplakin II promoter, Uroplakin Ia, Ib, II, and III, Desmin promoter, Elastase-1 promoter, Endoglin promoter, Flt-2 promoter, GFAP promoter, ICAM-2 promoter, INF-alpha promoter, INF-beta promoter, OG-2 promoter, SP-B promoter, Syn1 promoter, Albumin promoter, AFP promoter, CCKAR promoter, CEA promoter, c-erb2 promoter, COX-2 promoter, CXCR4 promoter, E2F-1 promoter, LP promoter, MUC1 promoter, Survivin promoter, TRP1 promoter, Tyr promoter, Uromodulin promoter, PCK1 promoter, CHDH promoter, ASPA promoter, PKLR promoter, TCF2 promoter, PKHD1 promoter, UPB1 promoter, SSTR1 promoter, HYAL1 promoter, FANCA1 promoter, KLRC3 promoter, KLRC2 promoter, APOBEC1 promoter, CEACAM1 promoter, GYS2 promoter, ADH4 promoter, ALB promoter, SFTPB promoter, PLUNC promoter, WISP2 promoter, PRLR promoter, WT1 promoter, PAEP promoter, FOLR1 promoter, VIT promoter, UCN3promoter, IPF1 promoter, INS promoter, CTRB1 promoter, S1 promoter, MAGEA4 promoter, Telomerase promoter, and the like.

In addition, the same or a different enhancer is used. The enhancer can include, but is not limited to. Prostate Specific Antigen enhancer, Prostate Specific Membrane Antigen enhancer, Probasin Enhancer, Prostate Stem Cell Antigen Enhancer, and the like.

### Example 6

### Development of Other Cancer-Selective CTC-RVs

Example 1 through 4 above are repeated using other cancer cell specific promoters and enhancers. The type of cancer can include, but is not limited to, small intestine cancer, bladder cancer, lung cancer, thyroid cancer, uterine cancer, liver cancer, kidney cancer, breast cancer, stomach cancer, testicular cancer, cervical cancer, esophageal cancer, ovarian cancer, colon cancer, melanoma, prostate cancer, and the like.

### Example 7

### Development of Other Cell Type Specific Reporter Vectors

Example 1 through 4 above are repeated using other cell type specific promoters and enhancers. The cell type can include, but is not limited to, cancer cell, a stromal cell, a mesenchymal cell, an endothelial cell, a fetal cell, a stem cell, a non-hematopoietic cell, and the like.

### Example 8

### Studies Using Multiple Cell Type Specific Reporter Vectors

Routine experiments are conducted to develop compositions and methods for using multiple cell type specific reporter vectors to probe a biological sample. It is expected that each reporter vector tested will show the sensitivity and specificity expected from the work performed in Examples 1 through 4.

### REFERENCES

1. Ashworth, T. R., 14 MED. J. AAUSTRALIA 146-69 (1869).
2. Jemal et al., 59(4) C A CANCER J. CLIN. 225-49 (2009).
3. Cohen et al., 26(19) J. CLIN. ONCOL. 3213-21 (2008).
4. Cristofanilli et al., 351(8) N. ENG. J. MED. 781-91 (2004).
5. Danila et al., 13(23) CLIN. CANCER RES. 7053-58 (2007).
6. Budd et al., 12(21) CLIN. CANCER RES. 6403-09 (2006).
7. de Bono et al., 14(19) CLIN. CANCER RES. 6302-09 (2008).
8. Brandt et al., 56(20 CANCER RES. 4556-61 (1996).
9. Racila et al., 95(8) PROC. NATL. ACAD. SCI. U.S.A. 4589-94 (1998).
10. Wang et al., 88(12) CANCER 2787-95 (2000).
11. Witzig et al., 8(5) CLIN. CANCER RES. 1085-91 (2002).
12. Austrup et al., 83(12) BR. J. CANCER 1664-73 (2000).
13. Vona et al., 156(1) AM. J. PATHOL. 57-63 (2000).
14. Ghossein et al., 13(5) J. CLIN. ONCOL. 1195-2000 (1995).
15. Schuster et al., 108(2) INT. J. CANCER 219-27 (2004).
16. Bossolasco et al., 26(1) CANCER DETECT. PREV. 60-3 (2002).
17. Xenidis et al., 24(23) J. CLIN. ONCOL. 3756-62 (2006).
18. Hardingham et al., 1(7) MOL. MED. 789-94 (1995).
19. Khan et al., 6(9) CLIN. CANCER RES. 3499-3504 (2000).
20. Smirnov et al., 65(12) CANCER RES. 4993-97 (2005).
21. Fehm et al., 8(7) CLIN. CANCER RES. 2073-84 (2002).
22. Wulfing et al. 12(6) CLIN. CANCER RES. 1715-20 (2006).
23. Cabioglu et al., 184(5) AM. J. SURG. 414-17 (2008).
24. Cruz et al., 123(1) AM. J. CLIN. PATHOL. 66-74 (2005).
25. Pachmann et al., 26(8) J. CLIN. ONCOL. 1208-15 (2008).
26. Wrage et al., 15(5) CLIN. CANCER RES. 1566-74 (2009).
27. Mueller et al., 83(3) CANCER 538-46 (1998).
28. Soeth et al., 57(15) CANCER RES. 3106-10 (1997).
29. Leers et al., 55(5) J. CLIN. PATHOL. 359-66 (2002).
30. Berger et al., 41(5) EUROPEAN UROLOGY 532-39 (2002).
31. Ahrendt et al., 91(4) J. NATL. CANCER INST. 332-39 (1999).
32. Ghossein et al., 5(8) CLIN. CANCER RES. 1950-60 (1999).
33. Went et al., 35(1) HUM. PATHOL. 122-28 (2004).
34. Fields et al., 2(9) FIELDS VIROLOGY 3091 (5^{th} Ed., Philadelphia: Wolters Kluwer Health/Lippincott Williams & Wilkins (2007)).
35. Rodriguez et al., 57(13) CANCER RES. 2559-63 (1997).
36. DeWeese et al., 61(20) CANCER RES. 7464-72 (2001).
37. Li et al., 55(1) INT. J. RADIAT. ONCOL. BIOL. PHYS. 204-14 (2003).
38. Small et al., 14(1) MOL. THER. 107-17 (2006).
39. Yu et al., 61(2) CANCER RES. 517-25 (2001).
40. Yu et al., 59(17) CANCER RES. 4200-03 (1999).
41. Lupold et al., 35(20) NUCLEIC ACIDS RES. e138 (2007).
42. Thomas and Mathews, 22 CELL 523-33 (1980).
43. Hoti et al., 15(8) MOL. THER. 1495-1503 (2007).
44. Leon et al., 95(22) PROC. NATL. ACAD. SCI. U.S.A. 13159-64 (1998).
45. Chiarugi and Giannoni, 76(11) BIOCHEM. PHARMACOL. 1353-64 (2008).
46. Glinsky et al., 5(2) CELL CYCLE 191-97 (2006).
47. Pfitzenmaier et al., 25(3) UROL. ONCOL. 214-20 (2007).
48. Fong et al., 146(3) SURGERY 498-505 (2009).
49. Kojima et al., 119(10) J. CLIN. INVEST. 3172-81 (2009).
50. Hoti et al., 14(6) MOL. THER. 768-78 (2006).
51. Rodriguez et al., 74(9) APPL. ENVIRON. MICROBIOL. 2583-87 (2008).
52. van der Poel et al., 168(1) J. UROL. 266-72 (2002).
53. van der Poel et al., 8(12) CANCER GENE THER. 927-35 (2001).
54. Lupold et al., 62(14) CANCER RES. 4029-33 (2002).
55. Fallaux et al., (13) HUM. GENE THER. 1909-17 (1998).
56. Li et al., 62(9) CANCER RES. 2576-82 (2002).
57. Ribas et al., 69(18) CANCER RES. 7165-69 (2009).
58. Markova et al., 279(5) J. BIOL. CHEM. 3212-17 (2004).
59. McPherson et al., HENRY'S CLINICAL DIAGNOSIS & MANAGEMENT BY LABORATORY METODS 21^{st} Ed., Philadelphia: Sauders Elsevier (2007).
60. Allan and Keeney, 2010 J. ONCOL. 426218 (2010).
61. Mononen et al., 60(22) CANCER RES. 6479-81 (2000).
62. Sun et al., 25(28) ONCOGENE 3905-13 (2006).
63. Taplin et al., 332(21) N. ENG. J. MED. 1393-98 (1995).
64. Ford et al., 170(5) J. UROL. 1817-21 (2003).
65. Koivisto and Rantala, 187(2) J. PATHOL. 237-41 (1999).
66. Linja et al., 61(9) CANCER RES. 3550-55 (2001).
67. Visakorpi et al., 9(4) NAT. GENET. 410-06 (1995).
68. Dehm et al., 68(13) CANCER RES. 5469-77 (2008).
69. Flu et al., 69(1) CANCER RES. 16-22 (2009).
70. Jagla et al., 148(9) ENDOCRINOLOGY 4334-43 (2007).
71. Mohler, J. L., 617 ADV. EXP. MED. BIOL. 223-34 (2008).
72. Scher and Sawyers, 23(32) J. CLIN. ONCOL. 8253-61 (2005).
73. Luo et al., 2(5) NAT. PROTOC. 1236-47 (2007).
74. Goodman et al., 18(6) CANCER EPIDEMIOL. BIOMARKERS PREV. 1904-13 (2009).
75. Helo et al., 55(4) CLIN. CHEM. 765-73 (2009).
76. Davis et al., 179 (6) J. UROL. 2187-91 (2008).

1. An adenovirus construct comprising:
a. a cell type specific promoter that drives adenoviral replication; and
b. at least one reporter gene incorporated into the viral Major Late Transcriptional Unit.

2. (canceled)

3. The adenovirus construct of claim 1, further comprising an enhancer operably linked to the cell type specific promoter.

4. The adenovirus construct of claim 1, wherein the cell type specific promoter is operably linked to the E1 gene.

5. The adenovirus construct of claim 1, wherein the cell type is selected from the group consisting of a cancer cell, a stromal cell, a mesenchymal cell, an endothelial cell, a fetal cell, a stem cell, and a non-hematopoietic cell.

6. The adenovirus construct of claim 1, wherein the cell type is a cancer cell.

7. The adenovirus construct of claim 6, wherein the cancer is selected from the group consisting of small intestine cancer, bladder cancer, lung cancer, thyroid cancer, uterine cancer, liver cancer, kidney cancer, breast cancer, stomach cancer, testicular cancer, cervical cancer, esophageal cancer, ovarian cancer, colon cancer, melanoma and prostate cancer.

8. The adenoviral construct of claim 1, wherein the reporter gene is a secreted reporter.

9. The adenoviral construct of claim 8, wherein the secreted reporter gene is selected from the group consisting of human chorionicgonadotrophin (hCG), alpha fetal protein (AFP), humanized Metridia luciferase (hMLuc), Gaussia Luciferase, Cypridina Luciferase, and Secreted Alkaline Phosphatase.

10. An adenovirus construct comprising:
a. a prostate cancer cell specific promoter that drives adenoviral replication; and
b. at least one reporter gene incorporated into the viral Major Late Transcriptional Unit.

11. (canceled)

12. The adenovirus construct of claim 10, wherein the prostate cancer cell specific promoter comprises prostate selective probasin promoter.

13. The adenovirus construct of claim 10, wherein the prostate cancer cell specific promoter is selected from the group consisting of Prostate Specific Antigen promoter, Probasin promoter, Prostate Specific Membrane Antigen promoter, Prostate Stem Cell Antigen promoter, Semenogelin promoter, KLK4 promoter, NKX3.1 promoter, AMACAR promoter, Uroplakin II promoter, Uroplakin Ia, Ib, II, and III, Desmin promoter, Elastase-1 promoter, Endoglin promoter, Flt-1 promoter, GFAP promoter, ICAM-2 promoter, INF-alpha promoter, INF-beta promoter, OG-2 promoter, SP-B promoter, Syn1 promoter, Albumin promoter, AFP promoter, CCKAR promoter, CEA promoter, c-erb2 promoter, COX-2 promoter, CXCR4 promoter, E2F-1 promoter, LP promoter, MUC1 promoter, Survivin promoter, TRP1 promoter, Tyr promoter, Uromodulin promoter, PCK1 promoter, CHDH promoter, ASPA promoter, PKLR promoter, TCF2 promoter, PKHD1 promoter, UPB1 promoter, SSTR1 promoter, HYAL1 promoter, FANCA1 promoter, KLRC3 promoter, KLRC2 promoter, APOBEC1 promoter, CEACAM1 promoter, GYS2 promoter, ADH4 promoter, ALB promoter, SFTPB promoter, PLUNC promoter, WISP2 promoter, PRLR promoter, WT1 promoter, PAEP promoter, FOLR1 promoter, VIT promoter, UCN3 promoter, IPF1 promoter, INS promoter, CTRB1 promoter, SI promoter, MAGEA4 promoter, and Telomerase promoter.

14. The adenovirus construct of claim 10, further comprising an enhancer operably linked to the prostate cancer cell specific promoter.

15. The adenovirus construct of claim 14, wherein the enhancer comprises prostate specific antigen enhancer.

16. The adenovirus construct of claim 14, wherein the enhancer is selected from the group consisting of Prostate Specific Antigen enhancer, Prostate Specific Membrane Antigen enhancer, Probasin Enhancer, and Prostate Stem Cell Antigen Enhancer.

17. The adenovirus construct of claim 10, wherein the cell type specific promoter is operably linked to the E1 gene.

18. The adenoviral construct of claim 10, wherein the reporter gene is a secreted reporter.

19. The adenovirus construct of claim 18, wherein the secreted reporter gene is selected from the group consisting of hCG, AFP, hMLuc, Gaussia Luciferase, Cypridina Luciferase, and Secreted Alkaline Phosphatase.

20. The adenovirus of claim 18, wherein the at least one secreted reporter gene expresses hCG, AFP, and/or hMLuc.

21. An adenovirus construct comprising:
a. prostate selective probasin promoter operably linked to the E1 gene; and
b. prostate specific antigen enhancer operably linked to the probasin promoter.

22. The adenovirus construct of claim 21, further comprising at least one reporter gene incorporated into the viral Major Late Transcriptional Unit.

23. The adenoviral construct of claim 22, wherein the reporter gene is a secreted reporter.

24. The adenovirus construct of claim 23, wherein the secreted reporter gene is selected from the group consisting of hCG, AFP, hMLuc, Gaussia Luciferase, Cypridina Luciferase, and Secreted Alkaline Phosphatase.

25. An adenovirus construct comprising a cell-type specific promoter operably linked to a reporter gene, wherein the reporter gene is inserted into the E1 gene.

26. (canceled)

27. A kit comprising the adenovirus of claim 25 and a helper virus.

28. A method for detecting circulating tumor cells in a biological sample from a patient comprising the steps of:
a. contacting the adenovirus construct of claim 7 with the biological sample obtained from a patient; and
b. analyzing reporter gene activity to detect circulating tumor cells in the biological sample.

29. A method for detecting circulating tumor cells in a biological sample from a patient comprising the steps of:
a. obtaining a biological sample from a patient, wherein the biological sample comprises a mixed cell population suspected of containing circulating tumor cells;
b. contacting the adenovirus construct of claim 7 with the biological sample; and
c. analyzing reporter gene activity to detect circulating tumor cells in the biological sample.

30. The method of claim 28, further comprising contacting the biological sample with a second adenovirus construct.

31. The method of claim 30, wherein the second adenovirus construct infects a different cell type than the first adenovirus construct.

32. A method for detecting circulating tumor cells in a biological sample from a patient comprising the steps of:
a. obtaining a biological sample from a patient, wherein the biological sample comprises a mixed cell population suspected of containing circulating tumor cells;
b. contacting the biological sample with a mixture of adenoviral constructs as in claim 7; and
c. analyzing reporter gene activity to detect circulating tumor cells in the biological sample.

33. A method for detecting a specific cell type in a biological sample from a patient comprising the steps of:
a. contacting the adenovirus construct of claim 1, with a biological sample obtained from a patient; and
b. analyzing reporter gene activity to detect the specific cell type in the biological sample.

34. The method of claim 28, wherein the biological sample is selected from the group consisting of whole blood, plasma, serum, urine, synovial fluid, saliva, tissue biopsy, surgical specimen, semen, and lavage.

35. A virus construct comprising:
a. a cell type specific promoter; and
b. at least one reporter gene incorporated into the viral Major Late Transcriptional Unit.

36. The virus construct of claim 35, wherein the virus is selected from the group consisting of adenovirus, herpes simplex virus, influenza virus, Newcastle disease virus, poliovirus, reovirus, vaccinia virus and vesicular virus.

37. (canceled)

38. (canceled)
